(19) United States
(12) Patent Application Publication
(10) Pub. No.: US 2012/0277171 A1
(43) Pub. Date: Nov. 1, 2012

(54) Warmi®, natural alternative for the treatment of menopause

(76) Inventor: Jose Luis Silva Martinot, Lima (PE)

(21) Appl. No.: 13/096,465

(22) Filed: Apr. 28, 2011

(51) Int. Cl. A61K 31/7048; A61P 25/24; A61P 3/06; A61P 25/22; A61P 15/00; A61P 5/30

(52) U.S. Cl. 514/24

(57) ABSTRACT

Warmi®, composed of glucosinolates, β-sitosterol and citrus flavonoids (hesperidin), is as effective as HRT in reducing menopausal symptoms, Warmi® promotes a healthy cardiovascular system, supports hormonal balance and emotional well-being, improving the quality of life of women during perimenopause and menopause. All of these and the lack of side effects make Warmi® an ideal natural alternative to conventional HRT.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO SEQUENCES LISTING, A TABLE, OR A COMPUTED PROGRAM LISTING COMPACT DISC APPENDIX

Not applicable.

### BACKGROUND OF THE INVENTION

The field of this invention pertains to class 424; Drug, Bio-Affecting and Body Treating Compositions specifically to “drug and bio-affecting compositions”. According to the U.S. patent classification definition this patent of utility and claimed invention are described as follows:

This class includes the following subject matter, not provided for elsewhere, when a utility set forth below is either (a) claimed or (b) solely disclosed;

A. Drug and bio-affecting compositions, which are generally capable of: (1) Preventing, alleviating, treating, or curing abnormal and pathological conditions of the living body, or (2) Maintaining, increasing, decreasing, limiting, or destroying a physiologic body function.

There comes a time in a woman's life when normal ovarian function gradually slows down, causing a sharp decline in estrogen production. This estrogen deficiency leads to irregular menstrual cycles and a number of short and long term symptoms. Among these are; vasomotor symptoms like hot flashes, sweating and insomnia as well as psychological disorders such as depression and mood swings. The genitourinary system is also affected, leading to urinary incontinence and urgency, UTIs and dyspareunia caused by atrophic vaginitis. Among the long term disorders associated with this condition are osteoporosis and cardiovascular disease.

The usual treatment for menopause is Hormone Replacement Therapy (HRT) and even though it does relieve many menopausal symptoms, particularly the vasomotor and urogenital, there appears to be an association between HRT and breast, endometrial and ovarian cancer, in addition to an increased risk of atherosclerotic events and cardiovascular disease. Approximately 70% of women abandon the treatment after the first year due to the adverse effects of HRT. These and other recent data on the use of conventional hormone therapies and their adverse affects have motivated women into considering other therapies. These alternative therapies include natural products such as phytoestrogens as well as SERMs; selective estrogen receptor modulators.

When it comes to natural alternatives evidence supporting their effectiveness is still lacking, and soy, a popular product for menopause, has also been associated to breast cancer, which is causing soy's popularity loss. Popular natural alternatives also include black cohosh, red clover, primrose seed oil, dong quai and ginseng. Several groups have concluded that these dietary supplements have limited or no beneficial effect on climacteric symptoms. It has been reported that most of these herbal preparations also present numerous and sometimes dangerous adverse effects. Warmi®, on the other hand, was envisioned as a “natural alternative to HRT without the side effects”, it was developed following all of the necessary preclinical and clinical evaluations to help support not only its efficacy but also show its safety.

Warmi® contains a natural proprietary blend composed of glucosinolates, β-sitosterol and citrus flavonoids (hesperidin). These phytochemicals have appreciable biological effects. Glucosinolates have been shown to induce cytoprotective enzymes, may be chemoprotective and promote a healthy cardiovascular system. β-sitosterol, the most common plant sterol, has become a useful dietary supplement for cholesterol management, it has also been reported that it has immune-modulating properties and may have a positive effect on the reproductive system due to its weak estrogenic activity. The flavonoids found on citrus fruits are known to be potent antioxidants. These appear to have beneficial effects on the cardiovascular system, may support bone metabolism and have chemoprotective properties. We believe that these compounds act synergistically giving Warmi® its beneficial effects.

### BRIEF SUMMARY OF THE INVENTION

When it comes to natural alternatives for hormone replacement therapy (HRT) evidence supporting their effectiveness is still lacking. Our product Warmi® has been evaluated in preclinical and clinical studies and these show that Warmi® not only relieves the symptoms associated with menopause, improving the quality of life of these women, but also that it's safe and free of the undesirable side effects normally associated with HRT.

Warmi® contains a natural proprietary blend composed of glucosinolates, β-sitosterol and citrus flavonoids (hesperidin). These phytochemicals have appreciable biological effects. Evidence shows they support bone metabolism and benefit the cardiovascular as well as the reproductive system, some of these compounds also have chemoprotective and immune-modulating properties.

Overall, our work shows Warmi® is as effective as HRT in reducing menopausal symptoms, Warmi® promotes a healthy cardiovascular system, supports hormonal balance and emotional well-being, improving the quality of life of women during perimenopause and menopause. All of these and the lack of side effects make Warmi® an ideal natural alternative to conventional HRT.

### BRIEF DESCRIPTION OF THE SEVERAL VIEWS OF THE DRAWING

Table 1. Baseline characteristics of randomized patients: sociodemographic, sexual and reproductive history.

Table 2. Anthropometric parameters of randomized patients.

Table 3. Effects on complete hemogram

FIG. 1. Warmi® does not have an effect on LH levels.

FIG. 2. Warmi® does not affect FSH levels significantly.

FIG. 3. Warmi® does not significantly affect dehydroepiandrosterone sulfate (DHEA-S) levels.

FIG. 4. Warmi® does not affect progesterone levels significantly.

FIG. 5. Warmi elevates estradiol levels after 3 months of treatment.

FIG. 6. Effects on glucose levels.

FIG. 7. Warmi reduces total cholesterol levels significantly after 6 months of treatment.

FIG. 8. Effects on triglycerides levels.

FIG. 9. Warmi elevates HDL levels significantly.

FIG. 10. Warmi decreases LDL levels significantly.

FIG. 11. Warmi decreases the CT/HDL ratio significantly after 3 months of treatment.

FIG. 12. Warmi decreases the LDUHDL ratio significantly after 3 months of treatment.

FIG. 13. Osteocalcin levels after 6 months of treatment.

FIG. 14. Warmi significantly reduces C reactive protein (CRP) levels after 6 months of treatment.

FIG. 15. Growth hormone levels after 6 months of treatment.

FIG. 16. Effects on interferon-gamma (IFN-γ) levels.

FIG. 17. Warmi significantly reduces IL-6 (interleukin 6) levels.

FIG. 18. Effects on tumor necrosis factor alpha (TNF-α).

Table 4. Distribution Frequency of PvuII and XbaI estrogen receptor polymorphism in a post-menopausal population of women in Peru

FIG. 19. Measurement of health parameters using the WHO Quality of Life BREF Questionnaire: Physical Health.

FIG. 20. Measurement of health parameters using the WHO Quality of Life BREF Questionnaire: Psychological.

FIG. 21. Measurement of health parameters using the WHO Quality of Life BREF Questionnaire: Social Relationships.

FIG. 22. Measurement of health parameters using the WHO Quality of Life BREF Questionnaire: Environment.

FIG. 23. Measurement of health parameters using the WHO Quality of Life BREF Questionnaire: All Parameters.

FIG. 24. Aspects of female sexuality measured using the McCoy Female Sexuality Questionnaire: Interest.

FIG. 25. Aspects of female sexuality measured using the McCoy Female Sexuality Questionnaire: Fantasy.

FIG. 26. Aspects of female sexuality measured using the McCoy Female Sexuality Questionnaire: Arousal.

FIG. 27. Aspects of female sexuality measured using the McCoy Female Sexuality Questionnaire: Satisfaction.

FIG. 28. Aspects of female sexuality measured using the McCoy Female Sexuality Questionnaire: Painful Intercourse.

FIG. 29. Aspects of female sexuality measured using the McCoy Female Sexuality Questionnaire: Orgasm Frequency.

FIG. 30. Aspects of female sexuality measured using the McCoy Female Sexuality Questionnaire: Vaginal Dryness.

FIG. 31. Measurement of anxiety using the Greene Climateric Scale.

FIG. 32. Measurement of depression using the Greene Climateric Scale.

FIG. 33. Measurement of somatic symptoms using the Greene Climateric Scale.

FIG. 34. Measurement of vasomotor symptoms using the Greene Climateric Scale. Data

FIG. 35. Measurement of sexual symptoms using the Greene Climateric Scale.

FIG. 36. Warmi®'s effect on MCF-7 cells (human breast adenocarcinoma cell line).

### DETAILED DESCRIPTION OF THE INVENTION

### Clinical Trial

Prospective, longitudinal, double blind, three stage, comparative clinical and laboratory evaluation trials on symptomatic postmenopausal women who attended the menopause medical units of the Hospital Nacional Cayetano Heredia, Hospital Nacional Arzobispo Loayza, Hospital Docente Madre Niño San Bartolomé and the medical unit of the Universidad Nacional Agraria La Molina.

The study was approved by the Ethics Committees of the National Hospitals Cayetano Heredia, Arzobispo Loayza and San Bartolomé and all the patients signed their consent prior to the commencement of the study. The study was conducted according to the Declaration of Helsinki III and the International Ethical Guidelines for Biomedical Research (Council for International Organizations of Medical Sciences—CIOMS/WHO).

### Treatment Groups:

Warmi® capsules (510 mg) were manufactured by Laboratorios Hersil S.A. Capsules were manufactured following Good Manufacturing Practices (GMP) and following the Hazard Analysis and Critical Control Points (HACCP). Warmi® is a natural proprietary blend composed of β-sitosterol, glucosinolates and citrus flavonoids (hesperidin). The product stability studies at 3, 6, 12, 15, 24 and 36 months show that these metabolites were chemically stable and meet the product standardization criteria. Patients received 3 capsules a day to be taken every 6 hours.

The placebo capsules were manufactured by Laboratorios Hersil S.A in a similar manner and appearance to the Warmi® capsules. The placebo capsule contained microcrystalline cellulose and magnesium stearate in the same quantity as Warmi®.

Tibolone was used as HRT. Tibolone was masked in a capsule with the same appearance as the previous groups. Patients received 3 capsules a day to be taken every 6 hours. Tibolone is the latest treatment for menopausal symptoms in Peru. Tibolone is a synthetic steroid with estrogenic, progestogenic and androgenic activity, used for the treatment of menopausal symptoms and post-menopausal osteoporosis. It has been observed that Tibolone reduces the level of triglycerides, HDL, total cholesterol and with no effect on the LDL.

### Sample Design:

The sample group was integrated by symptomatic postmenopausal women who attend the menopause centers of the participating hospitals. 135 participants were accepted in the study and each hospital recruited the patients that were randomly assigned to the three treatment groups: Warmi®, Tibolone and placebo.

A woman shall be considered menopausal if she has not menstruated for the last 12 or more months, with an FSH of between 25.0 and 134.8 mlU/ml, LH of between 7.7 and 58.5 mlU/ml, estradiol of between <10.0 and 39.5 pg/ml and progesterone of between 0.20 and 1.00 ng/ml. Prior to beginning the treatment and in each session of the study, each participant shall be evaluated by a quality of life questionnaire.

### Inclusion Criteria:

Symptomatic postmenopausal women aged 35 to 60.
Women who have moderate to intense menopausal symptoms.
Women with physiological or surgical menopause.
Women whose last menstruation was 12 months ago, prior to the start of the study.
Women who had not been taking HRT during for the past six months.
Women experiencing hot flashes.
Gynecologically healthy women, except for changes inherent to hypoestrogenism.
Women who voluntarily accept to participate in the study.

### Exclusion Criteria:

Asymptomatic postmenopausal women.
Women who have taken or used estrogens in the past 6 months.
Vegetarian women or women on a microbiotic diet.
Women who are smokers or alcoholics.
Asian women.
Women that use HRT or other hormone treatment.
Women who have a thyroid dysfunction that may interfere with the symptoms.
Women with chronic morbidity or prior psychiatric diseases.
Women who use Tamoxifen or antibiotics.
Women with intolerance to any of the products, malabsorption syndrome, kidney failure or obstructive hepatopathy.

### Elimination Criteria:

Women who take/use sexual hormones.
Women who abandon the treatment.

### Tests:

### Hormone Count:

Laboratory tests were requested prior to the beginning of the treatments (basal levels) and after 3 and 6 months. The levels of the following hormones were measured in the Laboratory of the Molecular Biotechnology Unit of the Research and Development Laboratories of the Universidad Peruana Cayetano Heredia: FSH, LH, estradiol, progesterone and DHEAS. The following normal menopausal values shall be considered: FSH of between 25.0 and 134.8 mlU/mL, LH of between 7.7 and 58.5 mlU/mL, estradiol between <10.0 and 39.5 pg/mL and progesterone between 0.20 and 1.00 ng/mL.

In order to determine the hormonal levels, a peripheral blood test was taken from each patient on an empty stomach and the serum FSH, LH, estradiol, progesterone and DHEAS levels were taken using the immunochemical luminescence method, following the standardized procedures of the laboratory.

### Lipids and Glucose Count:

The triglycerides (TG), total cholesterol (TC), HDL, LDL and serum glucose levels (Valtek S.A.) were measured based on a peripheral blood test prior to the start of the treatment, at months 1, 3 and 6 following treatment. TG, TC and HDL levels were measured with a MicroLab semiautomatic analyzer, through an enzymatic colorimetric technique, using the manufacturer's manual and procedures. Normal values shall be TC<200 mg/dL, HDL>35 mg/dL, LDL<130 mg/dL and TG<170 mg/dL.

### IL-6, TNF-α and INF-y, Osteocalcin, Growth Hormone and Quantitative C-Reactive Protein Count:

Peripheral blood samples were obtained in vacutainer test tubes with anticoagulant (Becton Dickenson, BD). The biological sample was previously centrifuged and stored at a −70° C. for the respective cytokine count. Type Th1: TNF-α, INF-y and Th2: IL-6 cytokines were counted. The manufacturer's instructions were followed and the cytokine concentrations were calculated according to the specifications (Pharmingen, BD). Cytokines were measured prior to and upon conclusion of the treatment.

Plasma osteocalcin was measured through the EASIA technique (Enzyme Amplified Sensitivity Immunoassay) using monoclonal antibodies against different epitopes of human osteocalcin (Biosource International, Inc., USA), with a sensitivity of 0.4 ng/mL. Human osteocalcin was measured at the beginning of the treatment and after 6 months of treatment, according to the manufacturer's instructions.

The growth hormone (GH) was measured through the immunoenzymatic technique, using anti-HGH polyclonal antibodies (Diagnostic Automatic Inc., USA) to quantitively determine HGH. The sensitivity of the test is 0.5 ng/mL.

C-reactive protein from human plasma (Diagnostic Automation Inc., USA) was tested with a very sensitive ELISA using a monoclonal antibody against a different antigenic determinant on the C-Reactive Protein molecule, with a sensitivity of 0.1 mg/mL. C-reactive protein (CRP) was measured prior to treatment and after 6 months of treatment, according to the manufacturer's instructions.

The levels of cytokines, osteocalcin, growth hormone and C-reactive protein were measured in the Laboratory of the Molecular Biotechnology Unit of the Research and Development Laboratories of the Universidad Peruana Cayetano Heredia (UPCH). All laboratory procedures were conducted in duplicate, standards were used to ensure the reproducibility and quality of the results.

### Genotype Determination:

The extraction of the genomic DNA was carried out on peripheral blood samples using the QIAamp DNA mini kit (Qiagen Ltd., USA). The polymorphisms of the □ and β estradiol hormone receptor, the vitamin D receptor, iNOS, cNOS and insulin were analyzed in the Molecular Biotechnology Unit of the UPCH, using the amplification technique of the PCR followed by enzymatic digestion with specific restriction endonucleases.

PvuII and XbaI polymorphisms were analyzed using the PCR-RFLP technique (polymerase chain reaction restriction fragment lengths polymorphism). A fragment of DNA of 1.372 kb that contains the two polymorphite sites were amplified using primers reported by the literature. The PCR was conducted with 30 cycles, according to the following steps: denaturation at 64° C. for 60 s, annealing at 50° C. for 60 s and extension at 72° C. for 90 s. The PCR products were digested with PvuII and Sabih restriction endonucleases (Promega, Madison, USA). The digestion products were analyzed in 2% agarose gels and stained with ethidium bromide. The Pp heterozygous genotype exhibits fragments of 1372, 936 and 436 bp long and the Xx heterozygous genotype exhibits fragments of 1372, 982 and 390 bp long. P or X in capital letters represents the absence of restriction sites, while the p or x in small letters indicate the presence of restriction sites.

### Statistical Analysis:

The statistical analysis was done using a SPSS statistical package (version 11.0). The results were expressed as a mean±standard error (SE), and a p<0.05 was considered significant. The variables were analyzed as a change in relation to the basal level and compared by treatment groups.

### Results:

The basal demographic parameters of the randomized postmenopausal women who completed the study are shown in Table 1. There were no differences between the groups at the beginning of the supplementation.

Table 2 shows the anthropometric characteristics and vital signs after six months of supplementation. In the Warmi® group there is a significant reduction in the respiratory rate after 6 months, while on the Tibolone group there appears to be a significant reduction on the abdominal perimeter.

Table 3 shows the results of the full blood test of the patients. There was a small decrease in the hematocrit (1.43%) of the placebo group. Likewise, in the Tibolone and Warmi® groups, there is a slight increase of 3.53% and 0.32% in the hematocrit, respectively. Upon observing the other quantifiable parameters, no significant changes are noticed.

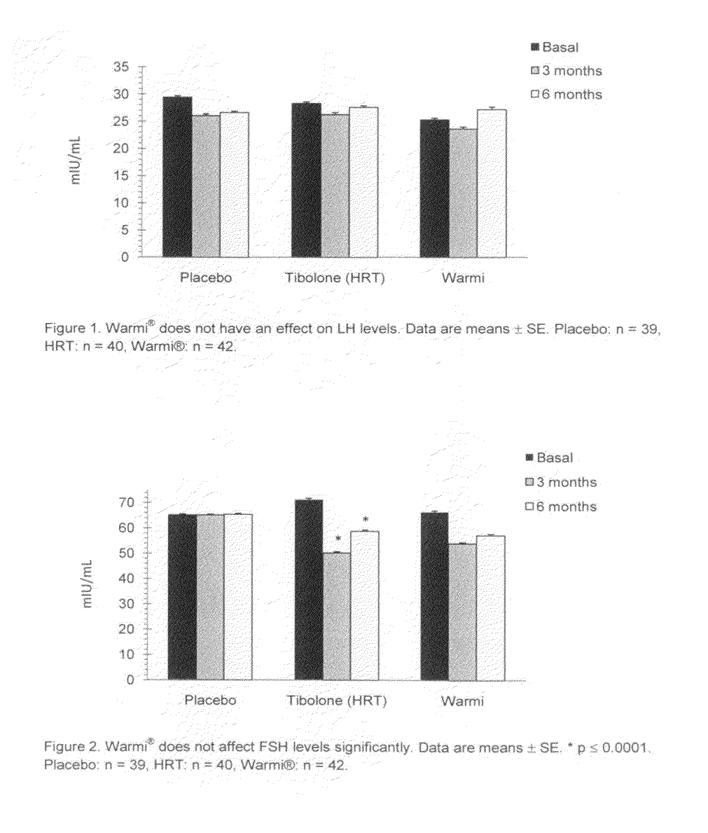

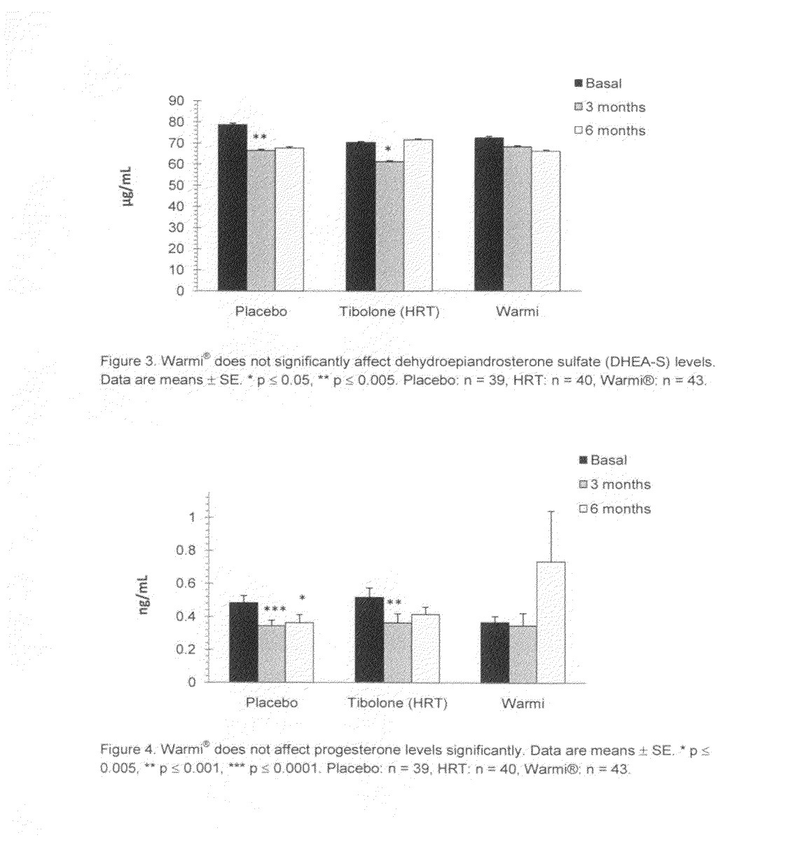

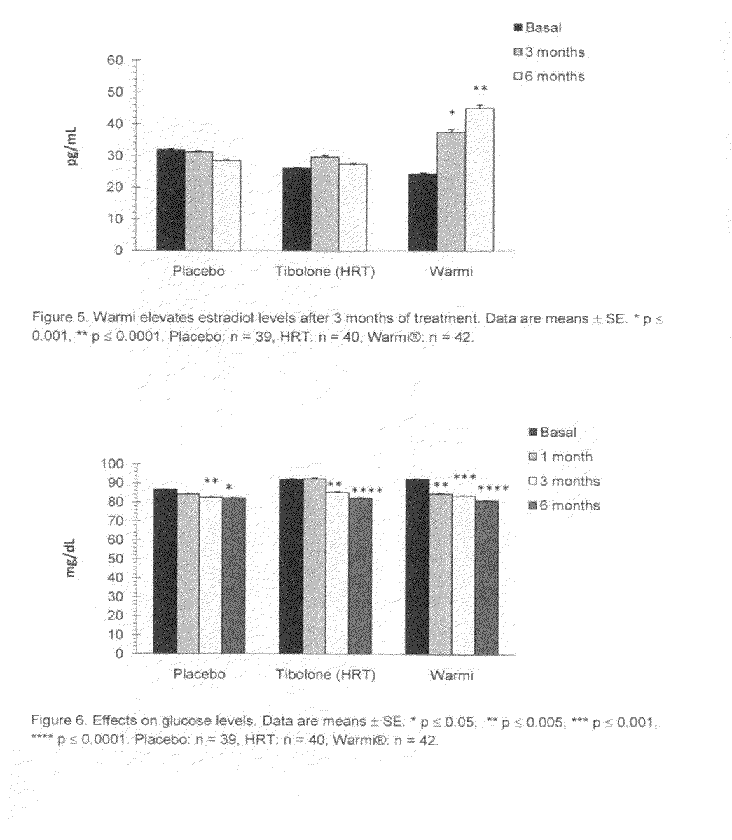

The LH levels are not significantly affected in any of the groups (FIG. 1). Upon analyzing FSH, we find that Tibolone shows a significant reduction of 29.58% at month three and 16.20% at month sixth of the treatment; while the Warmi® and Placebo didn't show any significant effects (FIG. 2). In the Placebo group, the DHEAS in serum values drop 15.49% at month three and 13.97% at month six of the treatment, while in the Tibolone group, they drop 12.87% at month three. In the Warmi® group there isn't a significant difference in FSH levels (FIG. 3). The measurement of the progesterone hormone shows that the Placebo group registers a decrease of 28.78% and 24.84% at month three and six respectively, the Tibolone group registers 29.70% at month three, while the Warmi® group shows no significant difference, although the is a positive trend after 6 months of treatment (FIG. 4). With respect to the estradiol (estrogen), only the Warmi® group showed a significant increase of 53.91% at month three and 84.99% at month six of the treatment was observed (FIG. 5).

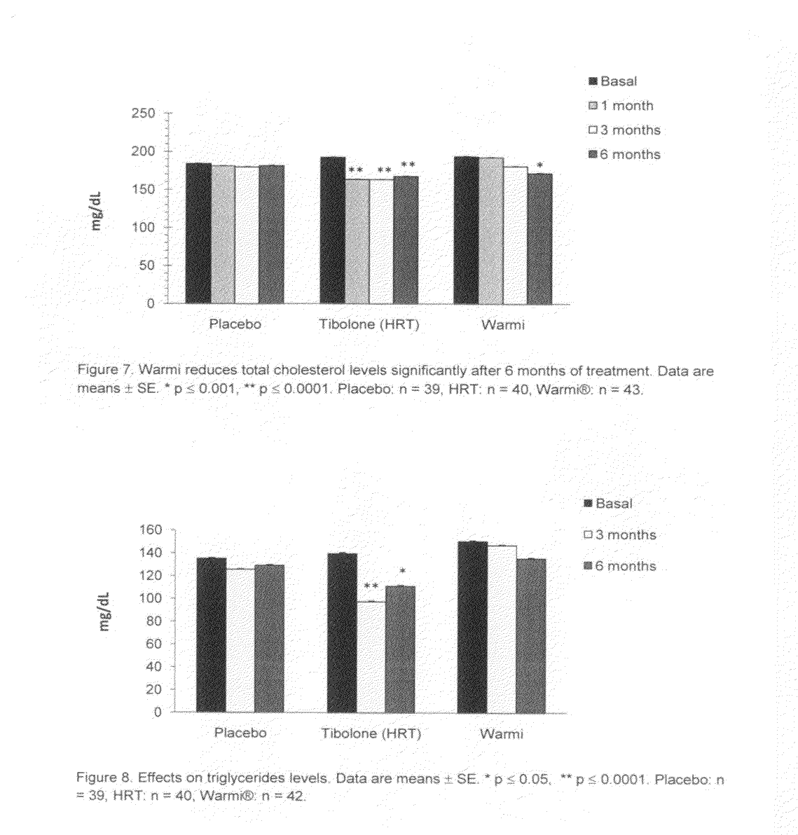

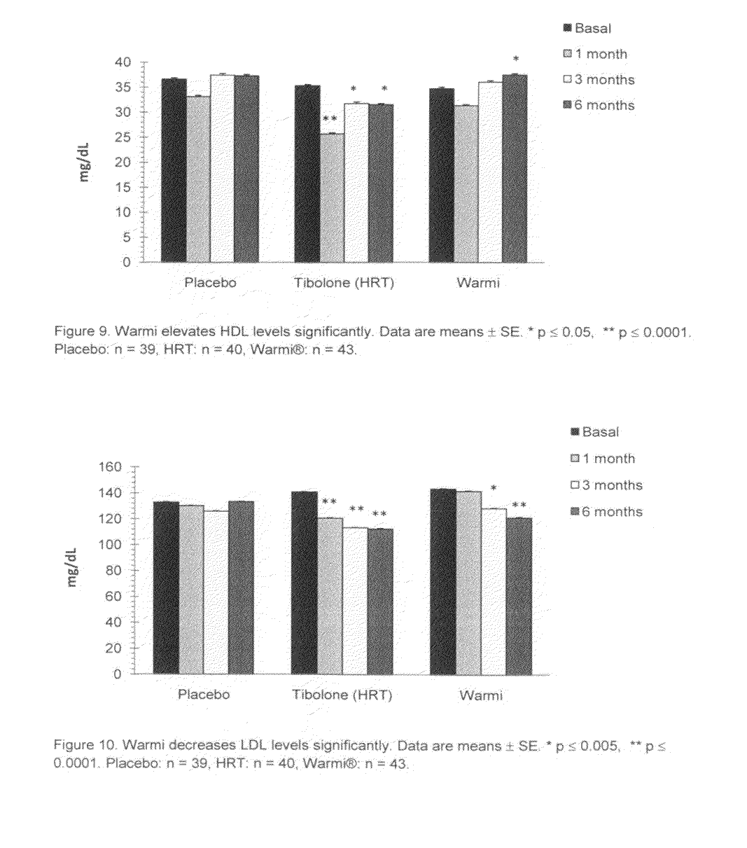

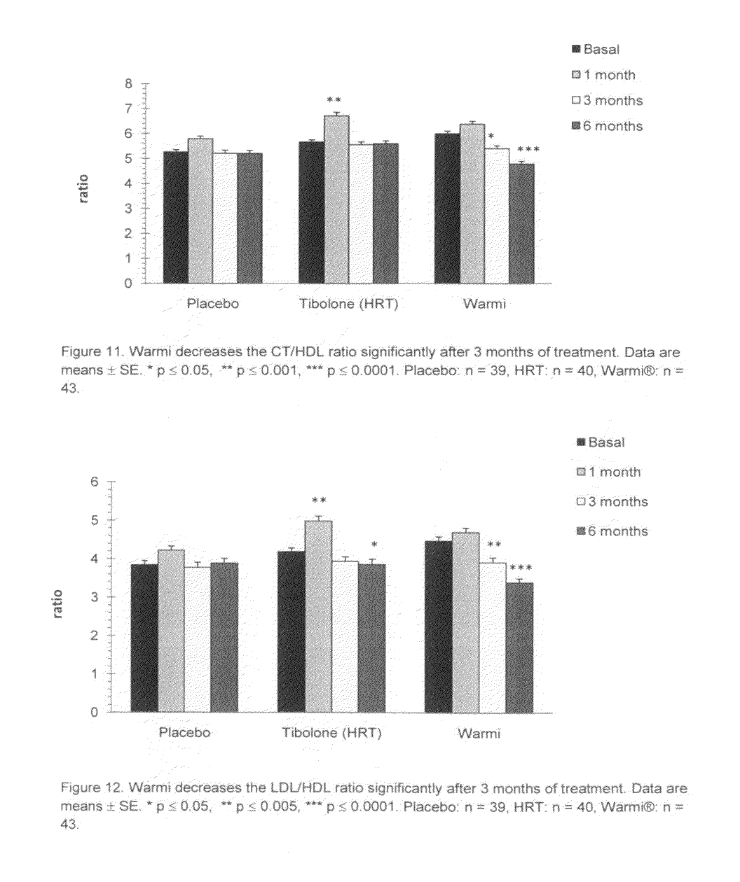

With regards to the blood glucose levels, it was observed that at the end of the study, the three treatment groups register a reduction in the level of glycemia of 3.54% for the Placebo group, 7.39% for the Tibolone group and 12.45% for the Warmi® group (FIG. 6). Upon analyzing the lipid profile values, it is observed that the basal values are homogenous in all the groups. A reduction in the cholesterol levels is registered mainly in the Tibolone and Warmi® groups. In terms of total cholesterol, after six months of treatment the Tibolone and Warmi® groups registered the greatest reduction, of 12.37% and 9.32%, respectively (FIG. 7). When the levels of triglycerides were evaluated only a significant decrease was observed in the Tibolone group (FIG. 8). With regards to the HDL, the Tibolone group registered a significant reduction after 1, 3 y 6 months of treatment, while the Warmi® group registered an increase of 5.62% after six months of treatment (FIG. 9). For the LDL levels, both the Tibolone group as well as the Warmi group registered a significant reduction (FIG. 10). Upon analyzing the Total Cholesterol/HDL, we found that only the Warmi® group showed a significant reduction (FIG. 11). In terms of the LDL/HDL ratio, after six months of treatment, the Tibolone group had a reduction while Warmi® showed a significant reduction after both 3 and 6 months of treatment (FIG. 12).

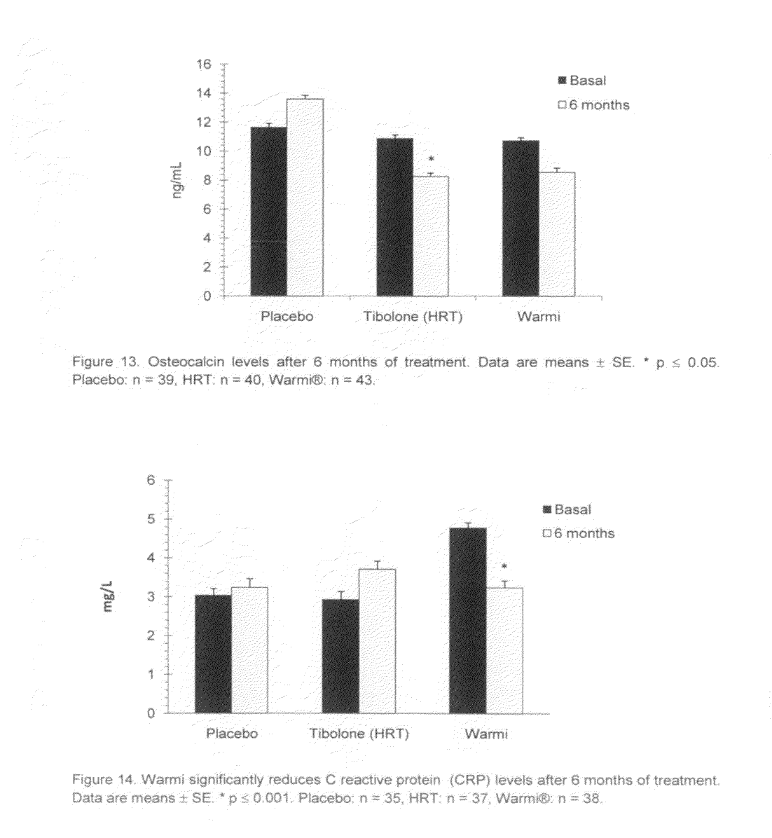

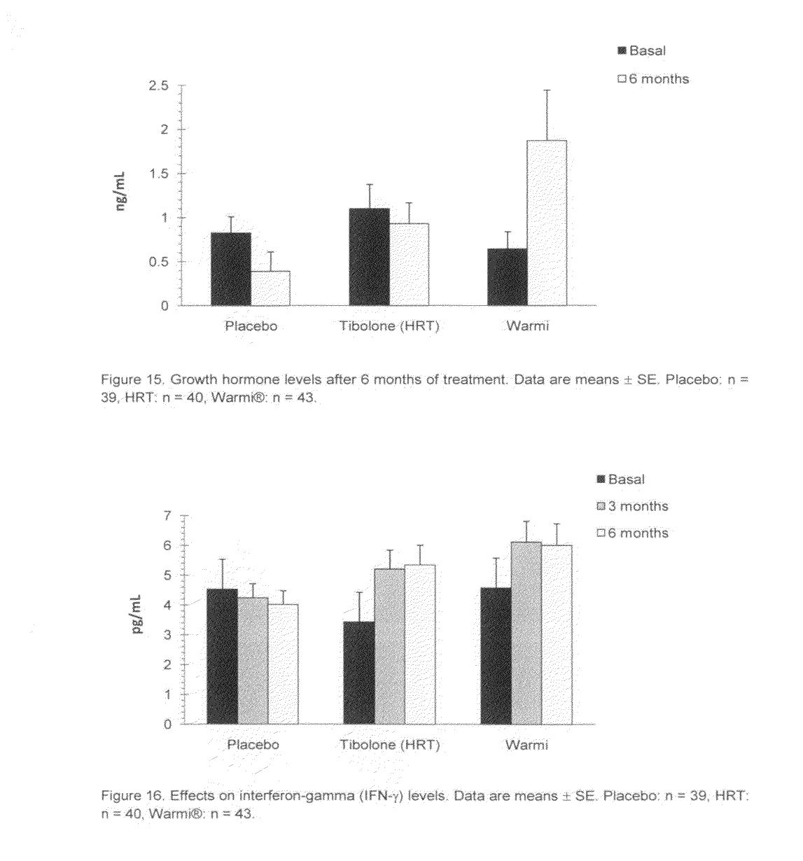

The results of the measurement of osteocalcin are shown in FIG. 13, where we find a significant reduction only on the Tibolone groups. With regards to the levels of the C-Reactive Protein (CRP), (FIG. 14) only the Warmi® group showed a significant reduction after 6 months of treatment. In relation to the changes in the percentage of CRP, a slight increase of 6.74% was observed in the Placebo group and of 26.83% in the Tibolone group, while a reduction of 32.14% was observed in the Warmi® group. When GH was evaluated no treatment showed any significant differences (FIG. 15).

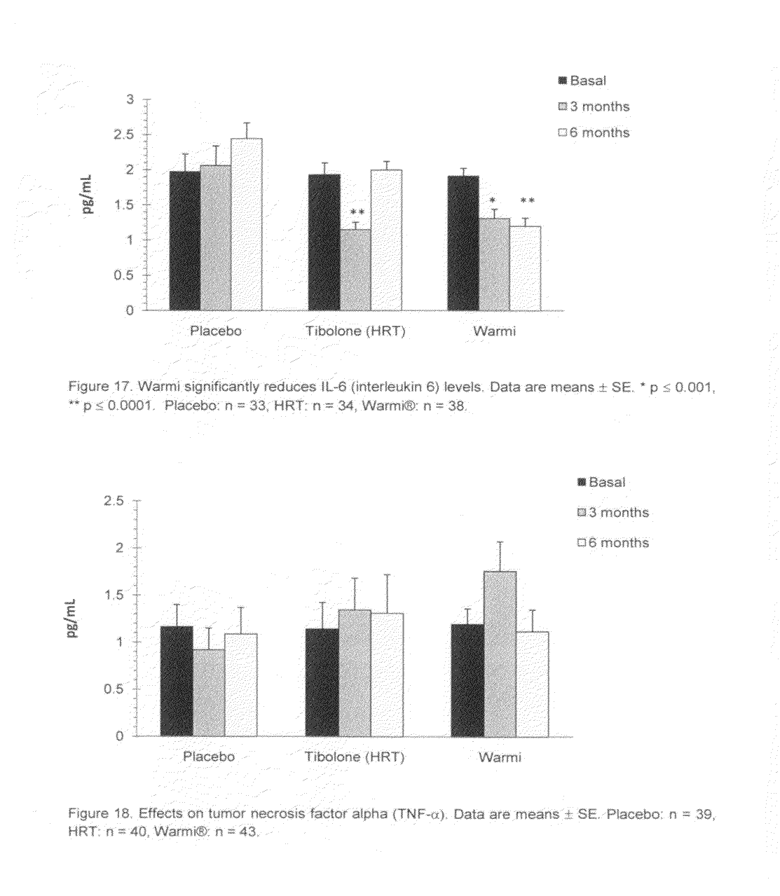

Upon analyzing some of the cytokines, we found that the postmenopausal women of the Tibolone and Warmi® groups register slight increases in the interferon gamma cytokine (INF-y) (FIG. 16) but none were significant. Interleukin 6 (IL-6) levels were significantly reduced in the Tibolone group after 3 months alone while in the Warmi® group a reduction after both treatment periods was observed (FIG. 17). Meanwhile, the results of the high Tumor Necrosis Factor (TNF-a show no significant effect on either of the groups (FIG. 18).

Warmi® improves the quality of life of the postmenopausal woman. Quality of life is defined, in general terms, as the wellbeing, happiness and satisfaction of an individual, thereby granting him/her the capacity to act, function or a positive feeling of his/her life. According to the WHO, quality of life is “an individual's perception of their position in life, in the context of the culture and value systems in which they live and in relation to his goals, expectations, standards and concerns. It is a very wide concept complexly influenced by their physical health, psychological state, level of independence, social relations, personal beliefs and their relationship to the salient features of the environment”.

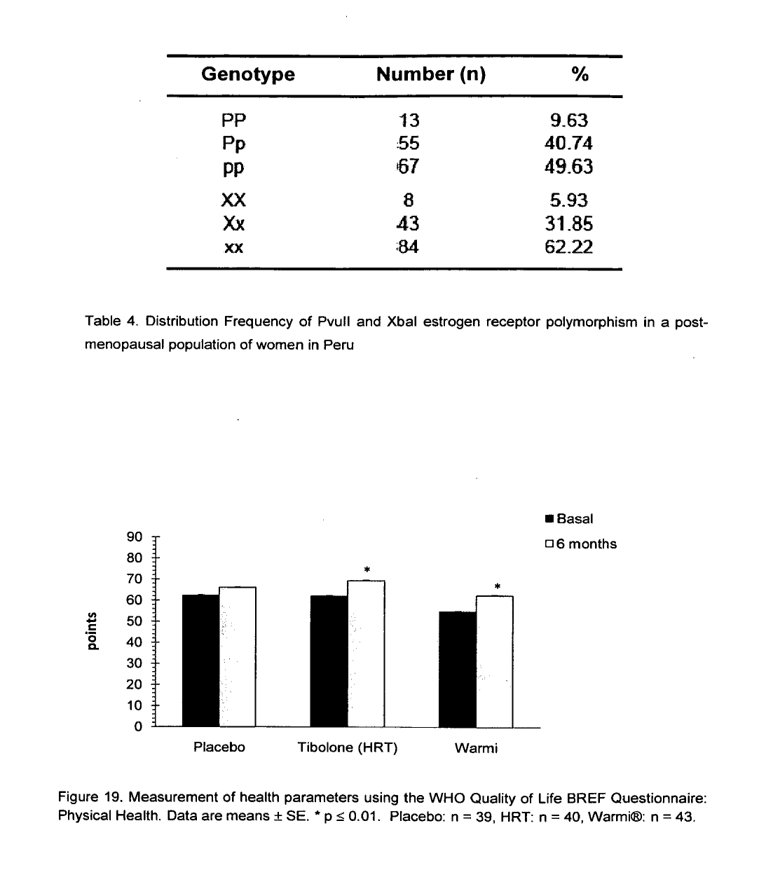

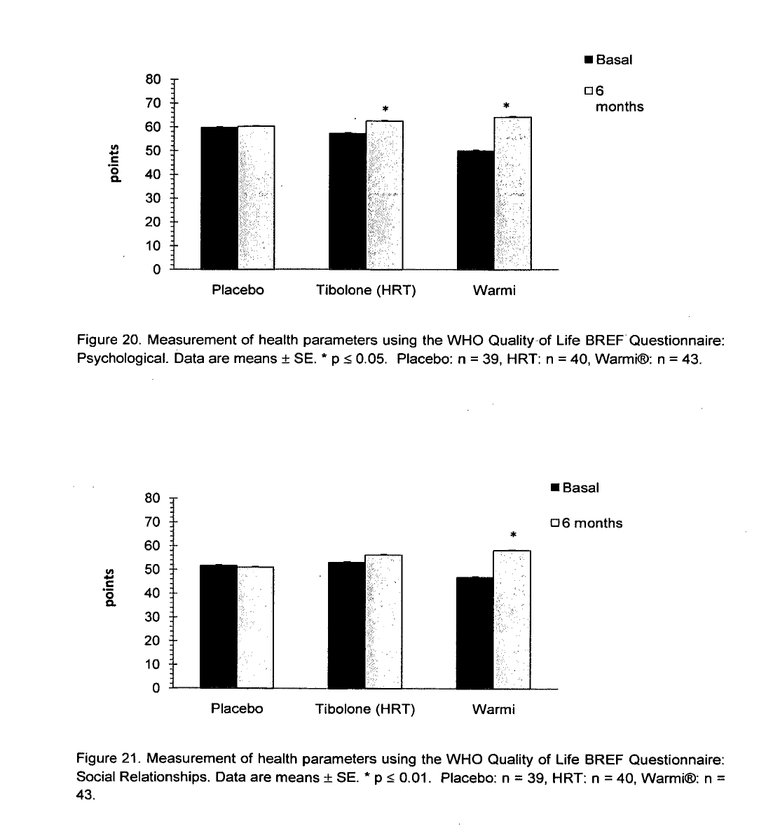

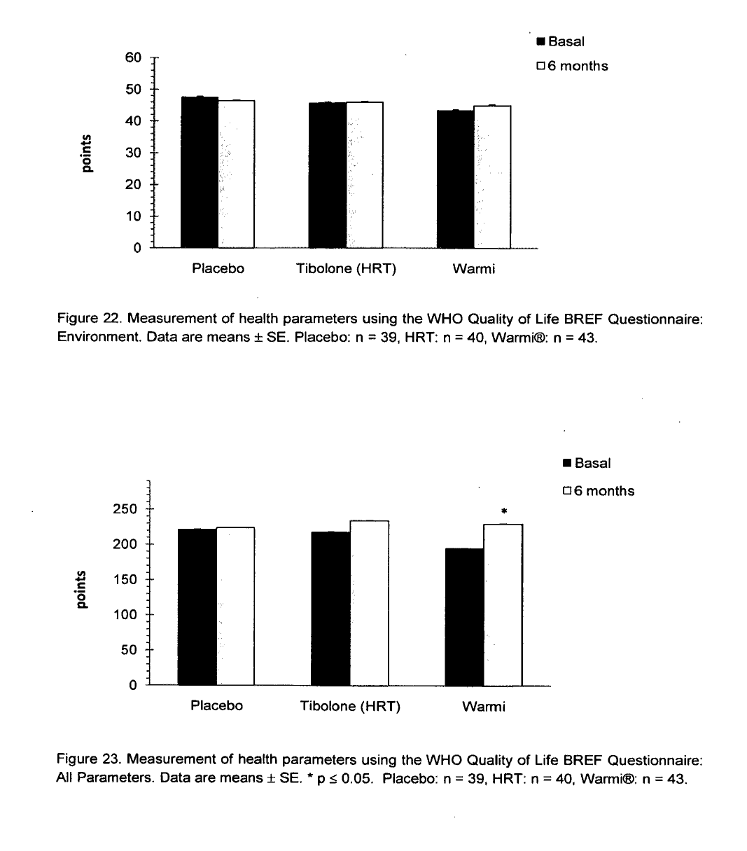

The WHOQOL-BREF is a survey that asks how the person feels about his quality of life, in terms of health or other areas of life, consisting of 26 items: 24 questions classified into 4 subscales/facets or domains, including physical health, psychological health, social and environmental relations. The higher the score, the more positive the quality of life (FIGS. 19-23).

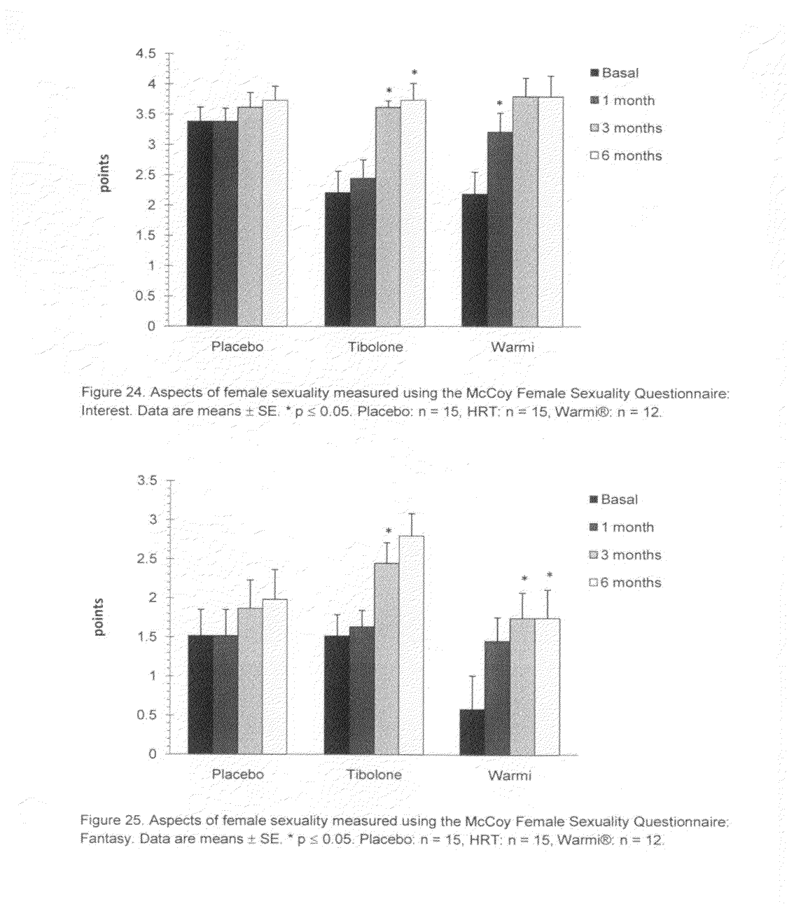

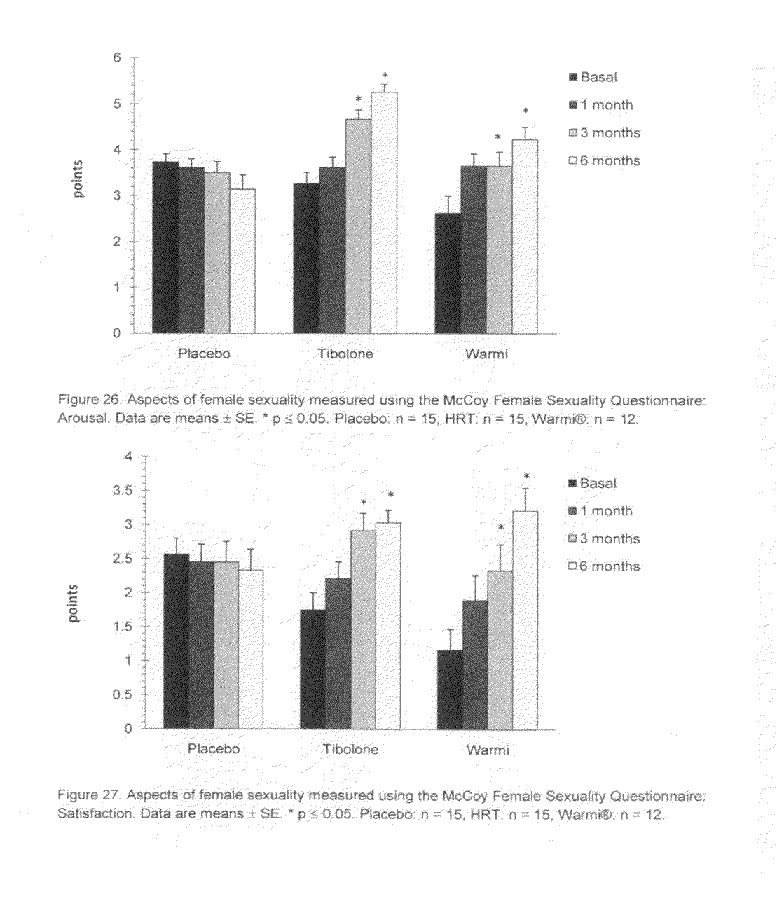

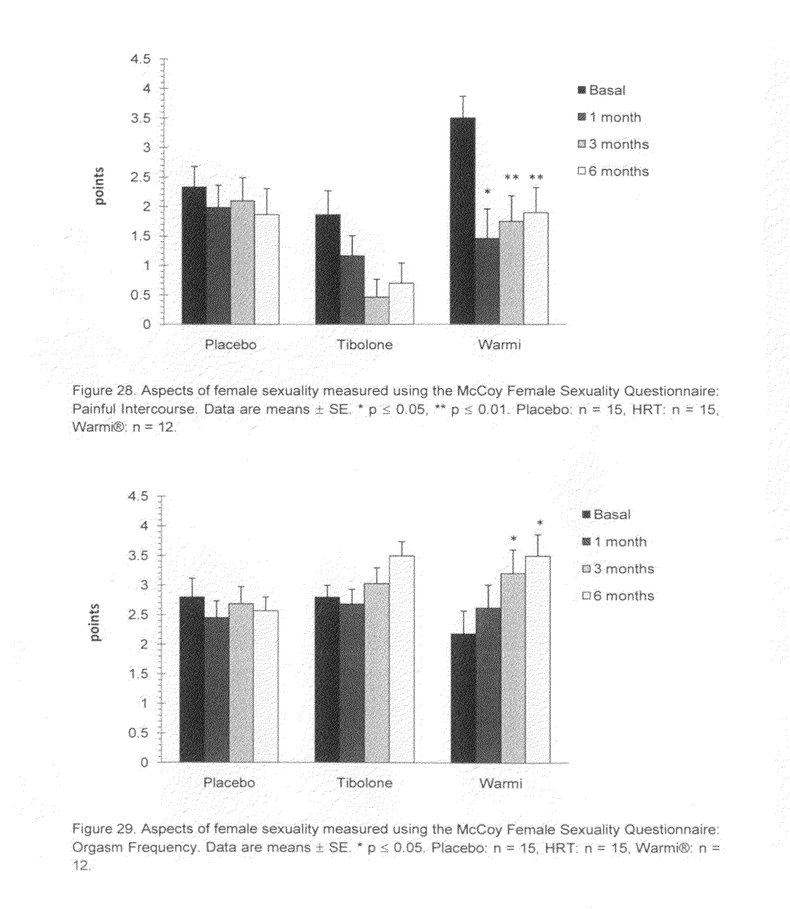

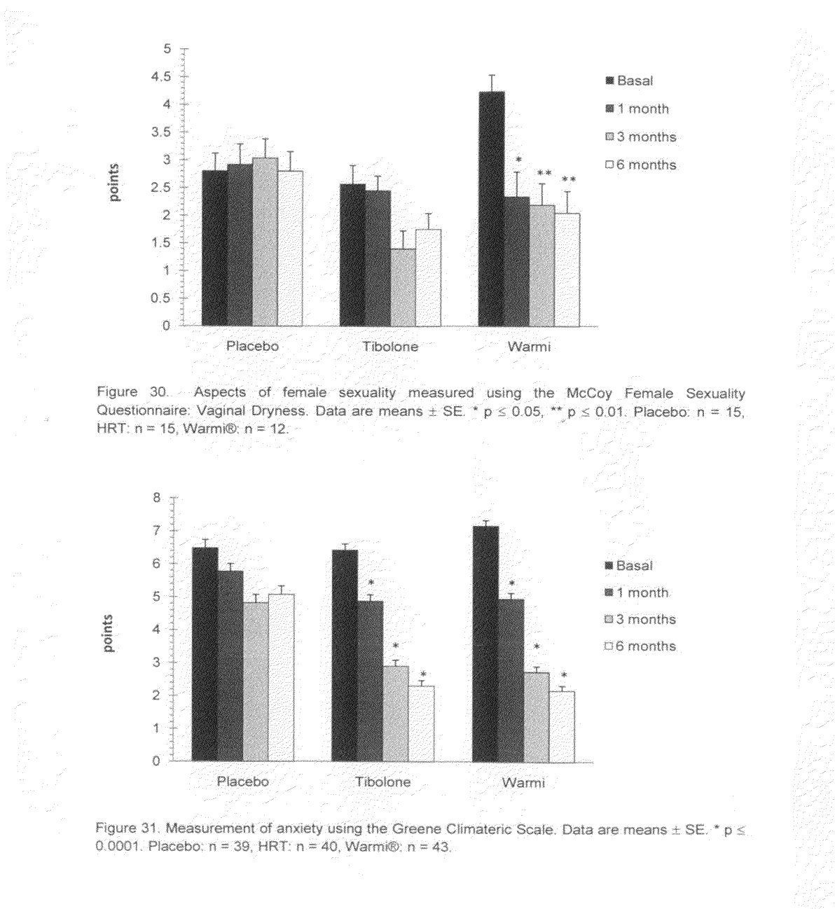

Sexual parameters were measured using the McCoy Female Sexuality Questionnaire, results show a significant increase in sexual interest, fantasy, arousal, satisfaction, orgasm frequency and a decrease in the incidence of painful intercourse and vaginal dryness, most of which were observed after 3 months of treatment (FIGS. 24-30).

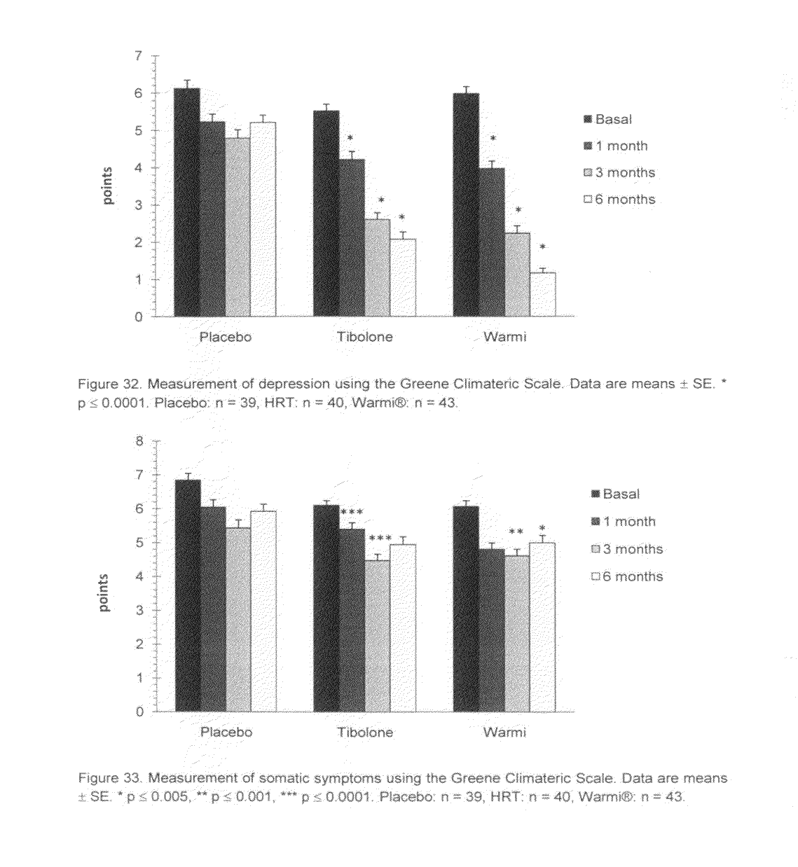

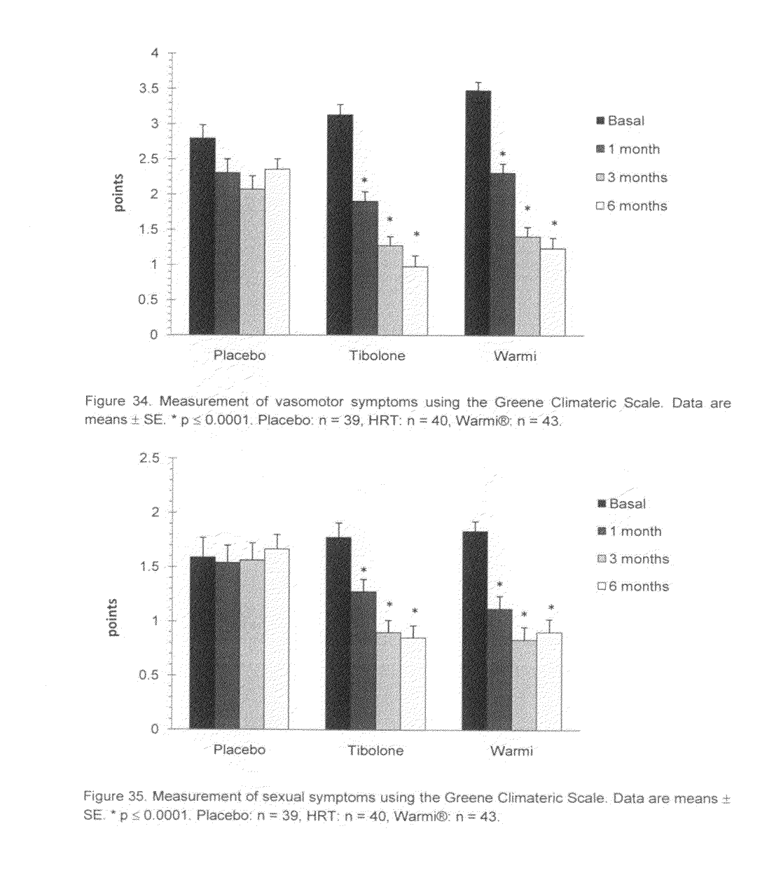

Changes were also observed in those patients who were supplemented with Tibolone Warmi® in the Green Survey (FIGS. 31-35). The changes are observed in sub-categories of Anxiety, Depression, Vasomotor Symptoms and Sexual Aspects.

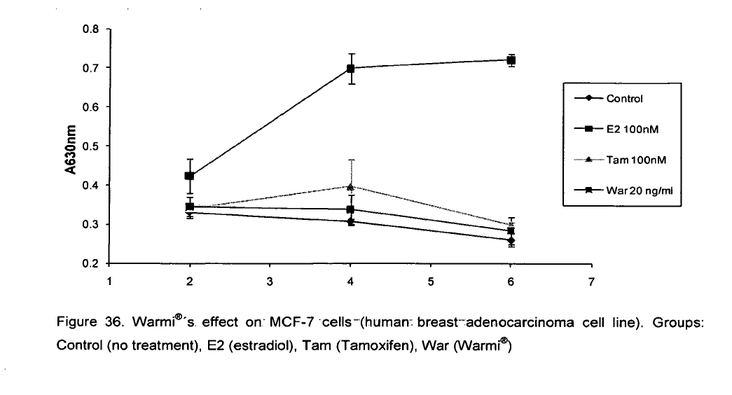

Given the fact that Warmi may have a weak estrogenic effect we did a preliminary preclinical evaluation of its proliferative effects (FIG. 36).

The figures indicate that polymorphism in the estrogen-a receptor can predict the levels of lipids, lipid response to hormone replacement therapy (HRT), the risk of heart attacks, the risk of bone fracture, bone mineral density (BMD) and constant changes in BMD. In this study, the distribution of the Era PvuII and XbaI genotypes were assessed first in 135 women who received HRT or the alternative to HRT (Tibolone, Warmi and Placebo). The polymorphism was determined by RFLP (PCR. Restriction fragment length polymorphism). The PvuII genotype was distributed as follows: PP 9.63%, Pp 40.74%, pp 49.63%. The frequency of the XbaI genotype was: XX 5.93%, Xx 31.85%, xx 62.22% (Table 4).

### Discussion:

There comes a time in a woman's life when normal ovarian function gradually slows down, causing a sharp decline in estrogen production. This estrogen deficiency leads to irregular menstrual cycles and a number of short and long term symptoms. Among these are; vasomotor symptoms like hot flashes, sweating and insomnia as well as psychological disorders such as depression and mood swings. The genitourinary system is also affected, leading to urinary incontinence and urgency, UTIs and dyspareunia caused by atrophic vaginitis. Among the long term disorders associated with this condition are osteoporosis and cardiovascular disease.

The usual treatment for menopause is Hormone Replacement Therapy (HRT) and even though it does relieve many menopausal symptoms, particularly the vasomotor and urogenital, there appears to be an association between HRT and breast, endometrial and ovarian cancer, in addition to an increased risk of atherosclerotic events and cardiovascular disease. Approximately 70% of women abandon the treatment after the first year due to the adverse effects of HRT. These and other recent data on the use of conventional hormone therapies and their adverse affects have motivated women into considering other therapies. These alternative therapies include natural products such as phytoestrogens as well as SERMs; selective estrogen receptor modulators.

Warmi®, a natural alternative to HRT, could be used to relieve the uncomfortable premenopausal and menopausal symptoms. We have shown through preclinical and clinical studies that Warmi® not only relieves the symptoms associated with menopause, improving the quality of life of these women, but also that it's free of the undesirable side effects normally associated with HRT.

The preclinical work done on ovariectomized mice show that Warmi® may have a weak estrogenic activity which may help maintain physiological conditions normally altered during menopause. The Phase I trial showed that Warmi® is safe and well tolerated. And when Warmi® was tested on post-menopausal women in this multicentric, randomized, double-blind, placebo controlled clinical trial, we confirmed the weak estrogenic activity (evident after 3 months of treatment). It is not clear whether Warmi® has a direct or indirect effect on estrogen levels, but what is clear is that the effect is weak. The normal ranges for estradiol are 0 to 30 pg/mL for post-menopausal women and when HRT is used estradiol levels can reach values between 18 and 361 pg/mL, depending on the hormones used. Physicians recommend to raise estradiol levels to a minimum of 40 to 50 pg/mL to prevent bone loss (60 pg/mL or higher is considered optimal) and with Warmi® the E2 levels go from 24.4 to 44.9 pg/mL, even though this increase is significant it is still in the recommended range.

Regarding the potential association between breast cancer and the estrogenic effect seen, we have evaluated the effects of Warmi® at a cellular level, on MCF-7 breast cancer cells. MCF-7 cells are sensitive to estrogens like estradiol and tend to proliferate after exposure. Tamoxifen is a selective estrogen receptor modulator and it's prescribed in hormone-sensitive breast cancer. Warmi® treatment does not show an proliferative effect, behaving in a similar manner to the control group. Warmi® acted like the control, it didn't show any proliferative effects on breast cancer cells, while estradiol showed increased proliferation as expected.

Warmi® also shows an important effect on cardiovascular risk markers like CRP (C reactive protein) as well as a positive effect on total cholesterol LDL and HDL levels, reducing the CT/HDL an LDL/HDL ratios when compared to basal levels. The depletion of estrogen associated with menopause causes alterations in the lipidic-lipoproteic metabolism which could lead to the progression of atherosclerosis and its complications therefore this effects on the lipidic profile and CRP have significant clinical value. Another marker that was significantly reduced was IL-6 (interleukin 6), important because elevated levels have been associated with hot flashes and increased bone resorption. Three questionnaires were done to help measure menopausal symptoms and quality of life, these were; the Greene Scale, the McCoy Female Sexuality Questionnaire and the WHOQOL-BREF. All three show a significant and positive effect on physical and psychological health (like depression and anxiety). Sexual parameters were also improved, showing a significant increase in sexual interest, fantasy, arousal, satisfaction, orgasm frequency and a decrease in the incidence of painful intercourse and vaginal dryness, most of which were observed after 3 months of treatment.

Overall, our work shows Warmi® is as effective and safe as Tibolone (commonly used in Peru to treat menopausal symptoms) in reducing menopausal symptoms, Warmi® promotes a healthy cardiovascular system, supports hormonal balance and emotional well-being, improving the quality of life of women during perimenopause and menopause. All of these and the lack of side effects make Warmi® an ideal natural alternative to conventional HRT.

### APPENDIX TO THE SPECIFICATION

TABLE 1
Baseline characteristics of randomized patients: sociodemographic,
sexual and reproductive history.

| | PLACEBO | TIBOLONE | WARMI |
|---|---|---|---|
| | (n = 39) | (n = 40) | (n = 43) |
| Age | 49.41 ± 3.44 | 49.95 ± 4.32 | 51.52 ± 3.93 |
| Level of Instruction (%) | | | |
| Elementary School | 5.9 | 25 | 9.5 |
| High School | 35.3 | 25 | 33.3 |
| College/Graduate School | 58.8 | 50 | 57.1 |
| Occupation | | | |
| Housewife | 29.4 | 40 | 23.8 |
| Other profession | 70.6 | 60 | 76.2 |
| Marital status (%) | | | |
| Married | 58.8 | 50 | 81 |
| Co-habitation | 23.5 | 30 | 14.3 |
| Single | 17.6 | 20 | 4.8 |
| Number of Children (%) | | | |
| None | 11.8 | 20 | 14.3 |
| One | 5.9 | 25 | 4.8 |
| More than one | 82.4 | 55 | 81 |
| Years of postmenopause | 2.88 | 5.33 | 4.76 |
| Age of menarche | 12.88 ± 1.69 | 12.15 ± 1.46 | 12.67 ± 255 |
| Age of menopause | 46.53 ± 3.84 | 44.62 ± 3.82 | 46.76 ± 3.85 |
| Age of first intercourse | 22.35 ± 5.65 | 21.5 ± 6.84 | 21.33 ± 3.86 |

Data are means ± SD

TABLE 2
Anthropometric parameters of randomized patients.

| | Basal | | | 3^{rd} Month | | |
|---|---|---|---|---|---|---|
| | Placebo | Tibolone | Warmi | Placebo | Tibolone | Warmi |
| Weight (kg) | 64.25 ± 10.07 | 66.1 ± 16.4 | 62.85 ± 5.89 | 63.23 ± 11.01 | 65.2 ± 16.28 | 63.75 ± 6.15 |
| Height (m) | 1.54 ± 0.05 | 1.55 ± 0.05 | 1.54 ± 0.04 | | | |
| Abdominal | 87.62 ± 10.32 | 93.25 ± 13.3 | 87.33 ± 7.11 | 86.37 ± 16.44 | 90.16 ± 15.52 | 87.05 ± 6.51 |
| Perimeter | | | | | | |
| Hip Perimeter | 103.42 ± 9.24 | 106.41 ± 10.81 | 103.88 ± 5.95 | 102.5 ± 11.92 | 105.13 ± 11.51 | 104.33 ± 6.91 |
| Heart Rate | 64 ± 1.63 | 64.67 ± 3.81 | 63.94 ± 4.03 | 63 ± 2 | 63.66 ± 1.96 | 64.77 ± 3.59 |
| Respiratory | 17.5 ± 1 | 18 ± 0 | 17.56 ± 1.6 | 16 ± 0 | 16.33 ± 0.98 | 16.44 ± 0.72 |
| Rate | | | | | | |
| Systolic BP | 102.5 ± 15 | 115 ± 8.36 | 110 ± 7.55 | 100 ± 8.16 | 113.33 ± 8.16 | 102.87 ± 11.1 |
| Diastolic BP | 66.25 ± 13.76 | 66.96 ± 9.72 | 72.5 ± 7.07 | 68.75 ± 6.29 | 72.5 ± 10.83 | 69.37 ± 10.03 |
| Pulse | 64 ± 2.3 | 64 ± 3.79 | 63.78 ± 3.9 | 63 ± 2 | 64 ± 1.78 | 64 ± 3.31 |
| Pressure | | | | | | |

| | 6th Month | | |
|---|---|---|---|
| | Placebo | Tibolone | Warmi |
| Weight (kg) | 63.5 ± 11.61 | 65.30 ± 14.33 | 63.43 ± 7.49 |
| Height (m) | | | |
| Abdominal Perimeter | 85 ± 14.89 | 89.75 ± 14.52* | 85.22 ± 7.04 |
| Hip Perimeter | 103 ± 12.68 | 105.25 ± 9.57 | 104.66 ± 5.27 |
| Heart Rate | 62.5 ± 1 | 62.67 ± 2.42 | 63.11 ± 2.66 |
| Respiratory Rate | 16 ± 0 | 16.16 ± 0.41 | 16 ± 0* |
| Systolic BP | 102.5 ± 12.58 | 110 ± 8.98 | 106.25 ± 8.76 |
| Diastolic BP | 67.5 ± 9.57 | 73.16 ± 11.4 | 68.75 ± 9.54 |
| Pulse Pressure | 62.5 ± 1 | 62.67 ± 2.42 | 63.11 ± 2.67 |

Data are means ± SD,
*p ≦ 0.05

TABLE 3
Effects on complete hemogram

| | | Basal | | | 1^{st} Month | | |
|---|---|---|---|---|---|---|---|
| | | Placebo | Tibolone | Warmi | Placebo | Tibolone | Warmi |
| Hematocrit: | Mean (SD) | 40.35 (2.09) | 40.31 (2.40) | 39.5 (2.03) | 40.36 (2.56) | 41.50 (2.07) | 39.56 (2.70) |
| | % change from Basal | | | | 0.52 | 1.151 | 0.152 |
| Hemoglobin: | Mean (SD) | 13.45 (0.96) | 13.42 (0.82) | 13.16 (0.95) | 13.52 (0.85) | 13.86 (0.95) | 13.52 (0.92) |
| | % change from Basal | | | | 0.52 | 1.270 | 2.736 |
| Leucocytes | Mean (SD) | 6.42 (2.18) | 6.51 (1.68) | 6.16 (1.7) | 5.45 (1.77) | 5.73 (1.24) | 5.69 (1.25) |
| (k 1000): | % change from Basal | | | | −15.11 | −12 | −7.63 |
| Banded | Mean (SD) | 0.29 (0.58) | 0.33 (0.48) | 0.25 (0.44) | 0.53 (0.79) | 0.33 (0.59) | 0.6 (0.82) |
| Neutrophils: | % change from Basal | | | | 82.76 | 0 | 140 |
| Segmented | Mean (SD) | 37.62 (3.32) | 38.01 (2.3) | 38.73 (3.13) | 36.94 (4.42) | 28.44 (2.91) | 38 (3.10) |
| Neutrophils: | % change from Basal | | | | −1.522 | −0.29 | 0.420 |
| Eosinophiles: | Mean (SD) | 3 (1.45) | 3.33 (0.07) | 3 (1.03) | 3.24 (1.03) | 3.44 (0.70) | 3.07 (0.96) |
| | % change from Basal | | | | 8 | 3.303 | 3.133 |
| Basophiles: | Mean (SD) | 0.18 (0.39) | 0.11 (0.32) | 0.063 (0.25) | 0.18 (0.39) | 0 (0) | 0.07 (0.25) |
| | % change from Basal | | | | 0 | −100 | 11.11 |
| Monocytes: | Mean (SD) | 3.23 (1.2) | 2.89 (0.83) | 3.06 (0.63) | 3.64 (0.79) | 3.56 (1.09) | 3.33 (1.04) |
| | % change from Basal | | | | 12.6 | 13.18 | 8.124 |
| Lymphocytes: | Mean (SD) | 35.47 (2.52) | 34.72 (2.69) | 34.88 (2.39) | 35.47 (3.42) | 34.22 (2.53) | 33.93 (2.08) |
| | % change from Basal | | | | 0 | −1.44 | −2.724 |

| | | 3^{rd} Month | | | 6th Month | | |
|---|---|---|---|---|---|---|---|
| | | Placebo | Tibolone | Warmi | Placebo | Tibolone | Warmi |
| Hematocrit: | Mean (SD) | 38.67 (2.31) | 41.51 (2.66) | 40.35 (2.47) | 38.77 (2.54) | 41.72 (2.29) | 38.63 (2.95) |
| | % change | −1.685 | 2.977 | 2.152 | −1.437 | 3.523 | 0.329 |
| | from Basal | | | | | | |
| Hemoglobin: | Mean (SD) | 13.23 (0.78) | 13.83 (0.78) | 13.45 (0.69) | 13.26 (0.85) | 13.91 (0.76) | 13.21 (0.07) |
| | % change | −1.636 | 3.055 | 2.204 | 0.76 | 3.651 | 0.38 |
| | from Basal | | | | | | |
| Leucocytes | Mean (SD) | 5.69 (1.39) | 6.14 (1.41) | 6.19 (1.68) | 5.32 (1.46) | 5.51 (1.17) | 5.56 (1.22) |
| (k 1000): | % change | −11.37 | −5.68 | 0.487 | −9.346 | −14.4 | −9.74 |
| | from Basal | | | | | | |
| Banded | Mean (SD) | 0.71 (0.61) | 0.38 (0.50) | 0.4 (0.63) | 0.5 (0.07) | 0.1 (0.65) | 0.5 (0.70) |
| Neutrophils: | % change | 144.8 | 15.15 | 60 | 72.41 | 51.52 | 100 |
| | from Basal | | | | | | |
| Segmented | Mean (SD) | 58.47 (2.74) | 38.88 (2.13) | 38.4 (2.33) | 38.57 (1.90) | [symbol] 2 0.07 (1.34) | 28.7 (2.31) |
| Neutrophils: | % change | 1.124 | 0.401 | −0.596 | 2.2 | 1.491 | 1.517 |
| | from Basal | | | | | | |
| Eosinophiles: | Mean (SD) | 3.20 (0.61) | 3.10 (0.01) | 3.13 (0.90) | 3.17 (0.71) | 3.20 (0.01) | 3.4 (0.51) |
| | % change | 0.667 | 4.2 | 4.333 | 5.657 | 1.2 | 13.33 |
| | from Basal | | | | | | |
| Basophiles: | Mean (SD) | 0 (0) | 0.13 (0.50) | 0.07 (0.26) | 0 (0) | 0 (0) | 0.1 (0.31) |
| | % change | −100 | 18.18 | 11.11 | −100 | −100 | 58.73 |
| | from Basal | | | | | | |
| Monocytes: | Mean (SD) | 3.47 (1.12) | 3.63 (0.71) | 3.27 (1.22) | 3.75 (0.75) | 3.78 (0.97) | 3.7 (0.94) |
| | % change | 7.43 | 25.61 | 6.861 | 16.1 | 30.8 | 20.92 |
| | from Basal | | | | | | |
| Lymphocytes: | Mean (SD) | 34.05 (3.28) | 33.81 (2.51) | 34.73 (3.21) | 32.91 (2.53) | 32.56 (2.73) | 32.6 (2.17) |
| | % change | −4.003 | −2.62 | −0.41 | −7.217 | −6.22 | −6.537 |
| | from Basal | | | | | | |

[symbol]

indicates data missing or illegible when filed

1. Warmi® is a natural alternative to Hormonal Replacement Therapy (HRT); it supports hormonal balance in menopausal women, particularly through a weak estrogenic activity.

2. Warmi® promotes a healthy cardiovascular system by reducing LDL, total cholesterol (TC), the TC/HDL ratio as well as C-Reactive Protein (CRP) levels and by elevating HDL-cholesterol levels.

3. Warmi® improves the quality of life during menopause by promoting emotional well-being (reducing mood swings, anxiety and depression), reducing vasomotor symptoms (hot flashes) and improving sexual symptoms (reduces painful intercourse and vaginal dryness, it also increases arousal, sexual fantasy, orgasm frequency and sexual satisfaction).

4. Warmi® supports breast health and does not have a proliferative effect on human breast adenocarcinoma cells (MCF-7 cell line).
